(19) United States
(12) Patent Application Publication
(10) Pub. No.: US 2002/0139588 A1
(43) Pub. Date: Oct. 3, 2002

(54) Composite system for individual and group remote monitoring and directing of weight control procedures

(76) Inventor: Harvey Brandt, Arlington Heights, IL (US)

Correspondence Address: EVAN D. ROBERTS, 119 North Second Street, P.O. Box 369, Peotone, IL, 60468-0369, US

(21) Appl. No.: 09/820,362

(22) Filed: Mar. 29, 2001

(51) Int. Cl. G01G 19/40; A61B 10/00; G01G 19/00

(52) U.S. Cl. 177/25.16; 177/25.13; 702/173; 128/921

(57) ABSTRACT

A computerized composite system for individual and group remote monitoring and directing weight control procedures of human subjects. The system having a computerized central processing system with a software database and software management system to facilitate direct management and utilization of input data and responsively linked to one or more remote subject computer units having individual electronic weight scales with visual displays and being interfaced with the central processing system.

### BACKGROUND OF THE INVENTION

An individual interested in controlling their weight should have access to a complete weight control group and should be able to do so privately, while participating in such weight control. Unfortunately, the individual usually has to leave their home, or be concerned about inclement weather conditions or traveling from a remote location at a time that might not otherwise be convenient, and, which might not offer a weight control group. In addition, a member should not have to be concerned about having to attend a regular weight control group meeting at the same time on the same day each week.

Since people have started dieting, it has been necessary to develop and maintain some type of device and system to measure and control the progress or changes in an individual's weight. In the case of a person interested in changing or maintaining their weight or a dieter, the device has been a weight scale. It is vital to the person who is attempting to lose, gain or maintain their weight that they be able to view their weight results and note changes, if any, each time he or she uses the scale, during any interval, prescribed or at random, to measure these results. Whether a person, who is trying to control their weight has chosen a doctor, nutritionist, dietician, diet counselor or weight control group, a simple scale is used.

When dieters participate in any weight control program, whether through a group or one-on-one, the scale is the central measuring device used to view results. Even though many dieters might be participating in a diet or group therapy program, none have been able to include an important ingredient, namely, having their weight monitored by a scale that will report and facilitate recording their weight on a regular basis.

A dieter that has been on a periodic program would normally be required to weigh-in at the end of the period. Typically, at the same time each week If the dieter gets on their scale at home, just before attending their weekly meeting, and see a weight gain, the dieter might choose to skip the meeting. In considering the thought process of a dieter, if a dieter believes they have not performed well during the week, and knows for a fact that they have gained weight by weighing themselves at home, this becomes another reason for skipping a meeting and not attending their weekly weigh-in.

These facts not only hold true with a dieter participating in a weekly weight control group therapy session, but also for visiting a diet counselor, doctor or any type of diet consultant. A dieter sometimes assumes that they can skip one week and do better the next. Unfortunately, one missed week, more times than not, leads to another bad week and that leads to ultimate failure. Seeing negative unrecorded results on your usual scale at home could lead to ultimate failure on their diet.

Dieters choose to skip their group or individual sessions if they believe the assigned diet period has been unsuccessful. A dieter who is not sure that they have had a successful week does not want to face the very person who is supposed to be their support counselor or helper. They talk themselves out of attending their diet sessions. One missed week leads to another and to the ultimate failure. The scale at home may show a weight loss and the scale at their session may show a weight gain. This can cause apprehension and can also embarrass or otherwise discourage the dieter.

No matter what task an individual embarks on, the individual may think that he or she may fail. Individuals do not want to face a person who will chronicle their failure. Even though a person knows that a particular person is there to help, they still do not want to be a failure in front of that person or anyone else. Dieters do not like to deal with embarrassment or intimidation by having to face a person who is measuring their weight results when the dieter knows or feels they have had a bad diet period. Being observed face to face by a counselor or doctor or instructor, knowing that they have gained weight, is one of the main reasons why a dieter will more often than not skip a diet session.

A dieter participating on a weight control group or when working one-on-one with any dieter, doctor or counselor, needs the group therapy to be successful. The dieter will be most successful when the weight control group has at least regularly scheduled sessions designed to encourage them to lose weight, whether this session is one-on-one or a group session. For dieters to be successful, they must lose weight and keep it off. As long as they are being monitored and are losing weight on a regular basis, they will be successful. If the dieter stops losing, the dieter stops dieting because they are afraid to face a live monitor who will see them fail.

If a dieter is nominally fortunate enough to find an existing remote dieting program of some sort, the program will invariably incorporate one, or more, of the above negative aspects of dieting and not include certain beneficial and/or unique and novel dieting aids and controls that appear essential to overall consistent success in dieting.

### SUMMARY OF THE INVENTION

An illustrative example of the computerized composite system for individual and group remote monitoring and directing weight control of this invention is designated overall by the numeral 10 and includes, among other things, a computerized central management operational processing system (CMOPS) or central processing secondary system (CPS) 11, remote computers 12, electronic visual weight scales 13, and an initial application 14 of a subject human participant user 15 with an identification 16 and individual physiological profile 17 of subject user 15.

System 10 is a weight control and eating system which is operated thru a computerized internet 20 and educational group therapy software system 21 in central processing system 11. Applications 14 for participating subject users 15 are accepted from any qualified man, woman, adolescent or child interested in learning how to eat right and/or otherwise to lose weight and to keep it off. The system 10 requires that each individual subject user 15 have a smart scale 13, complete with a full readable digital display, its own privacy identification number 22 and compatible to interface with current computer systems.

Software 21 of central management operational processing system 11 collects, develops, compares and analyzes a library of individual profile 17 information from all participant users 15. This profile information 17 is used for the positive reinforcement and education of participants 15 for the purpose of directing that person in how to eat, exercise, foster and to promote good nutrition, offer related good health and diet information, and to aid in the education of dieting and maintenance of participant 15.

Included in CMOPS software 21 is private subject profile 17 developed for the use of each participant 14. At the same time, profile information 17 from the individual user 15 will be available to all users 15 on a generic basis as human portfolio 23 and as part of a profile library 18 for general user 15 information. All individual profile data 17 imputed to central processing system 11 will normally be available by privacy code only and only to member user 15 with the specific identification of individual member 15.

Central management processing system 11 software provides a generic diet system or specifically designed eating process, depending on the various user 15 requirements, to learn how to eat right and/or lose weight. Diet of individuals 15 will be altered in the software system 21 of central processor 11 and periodically updated based on the weekly results of individual user 15 and his or her requirements and the introduction of food quantity, variety and maintenance 26. Once participant 15 successfully completes a weight loss program they can continue within system 10 to participate and have access to an individualized process to retain the reduced weight of the user 15.

Software 21 of processing system 11 will receive, manage, analyze, compare, categorize and disseminate a variety of information used for the overall success and analysis of the program for its individual participants 15. Process system 11 has the software analytical ability and capacity, based on current technologies and new updated expanded technologies, including artificial intelligence, to oversee system 10. Further, system 10, through central process system 11, provides information to all members and will accumulate, store, and categorize all information received.

Processing system 11, of composite system 10, also provides an ongoing program for each individual profile 17 and expanded to include an entire group of participants 15 simultaneously. Each user 15 has a confidential secure program profile 17 which will monitor and accumulate data and results imputed into processing system 11. Simultaneously, the information will be imputed into a library or general human portfolio 23 of central processor 11 for all users 15 to view for the purpose of comparative analysis for the benefit of all users 15.

Although portfolio 23 data is taken from the individual privacy program profile 17, it is made generally available to the participants 15 in human portfolio 23 without disclosing or identifying any individual participant 15. All data 23 is listed in a general format by a user identification number 22 or other privacy identification number in order to maintain the privacy or secrecy of each participant 15.

Central processing system 11 receives data from each participant 15, including their weight report, from a weigh-in on a scale 13, from established report periods. Central processor 11 will take the data and weight received from a scale 13 into the system and add this data to an individual user 15 file and maintain a running record of the results. Certain programs are established, once a person becomes a user 15, and the programs are added as information becomes available thru the central processing system 11.

An individual profile 17 is established once a user has submitted application 14 and their use has been accepted into system 10. Application 14 will become the initial building block of profile 17. Additional data, as may be needed for profile 17, will be included from individual users 15 as it becomes needed and is available. In this regard, data will be obtained from weekly weigh-in results of the users from a scale 13, from surveys, recipes and places such as e-mails, suggestions and any additional user input from any interaction with central processor 11.

A before picture can be included in profile 17 of each participant 15 and can be continuously updated to reflect the results of the diets that participants 15 have participated in. Individual profile 17 will include all information or data about the participation of the individual participant 15, including favorite foods, weekly losses, goal weight, maintenance 26. Individual profile 17 will only be accessible by an individual participant 15 on an individual basis.

Group, general or human, portfolio 23 is generated by software 21 of processor 11. Information data will be taken from individual participant profiles 17 and categorized and made available for comparison to other individual profiles 17 within processor 11. General portfolios 23 form a library or encyclopedia of information in processor 11 where participants 15 can compare their results and progress with other participants 15 via system 10 and review a myriad of information pertaining to weight control.

Human portfolios 23 tabulate results according to categories, designed for comparison by age, gender, amount of exercise, prescribed or done, weight loss and weekly results of each participant 15. Each individual profile 17 will be listed in the human portfolio 23 screened by the privacy code 24 to maintain the secrecy and privacy of all participants 15 while allowing comparison for information and incentives between participants on portfolio 23.

Computerized central management and operational processor 11 continuously adds data allowing participant 15 to review and compare from any one weigh-in prior to another. Central processor 11 will also adjust diets as necessary to maintain the flexibility necessary to accommodate each participant 15. For example, it is not necessary that only a weekly weigh-in be submitted at the same time each week Participant 15 can weigh-in on a scale 13 at additional different times each week, if necessary, or as desired, and a weekly weigh-in would still be separately recognized and noted by processor 11 for systematic analysis. Any weigh-in for each participant 15, when reported, will be recognized by central processor 11. Weigh-in information from a scale 13 is automatically put into the individual participant profile 17 and also transferred into human general portfolio 23 for general screened review by other participants 15.

Processor 11 will develop a flow chart 25 to be established and updated on a regular basis for the purpose of maintenance 26 of development of participant profile 17. Composite system 10 will track results of individual participants 15 and also track the overall result of all participants 15. Central processor 11 will store data to help determine a system for each individual 15 to lose weight and will track the success of each member participant 15 based on the types of foods person 15 is eating. System 11 provides the strategy to develop a scheme that will compare larger weight losses with smaller losses and will interpret which foods have what kind of affect on the results of a participant 15 based on individual results of each participant 15.

Each smart scale 13 has its own computerized identification number which individualizes that particular scale 13 and ultimately identifies that scale 13 to the participant 15 that owns it. Each scale 13 has its own display showing the participant 15 their weight each time they get on scale 13. Many scales, or types of scales, for a variety of reasons, have a tendency to weigh differently than other similar scales. By having each participant 15 weighing themselves on their individual scale 13 they will always see a relatively consistently weight each time.

Participant 15 may choose to weigh themselves at anytime with the option of viewing their weight and having it reported and recorded into the processing system 11, or they may choose to just view their weight without having it reported and recorded. Scale 13 is connected directly into computer 12 and interacts directly through internet 20 via an interfacing connecting system into the software program of central processing system 11 and its hard drive system. This connection can be made via a wireless remote signal via satellite or other energy transmission.

All these above modes allow portable scale 13, to be transported by participant 15 to any location and will be able to transmit weight results, even with a different computer 12. Scale 13 and participant 15 will be identified either by a scale identification number 16 and/or a personal privacy code of participant 15 from any chosen location. Scale 13 is activated when user 15 checks their weight and chooses to have it transmitted to central processing system 11. That weight is then viewed by participating person 15 on computer 12 to allow participant 15 to confirm that their correct weight is being transmitted and is simultaneously sent via computer 12 to central processing system 11.

Software system of processor 11 functions to list categories and types of foods available. System 11 also lists all established nutrition values and allows the diet part of the program of system 10 to be available as an interactive process whereby the participant 15 may ask questions, choose or designate the types of foods they desire. Diet programs in processor 11 will also be able to make modifications for anything different in the schedule of a participant, such as eating outside the home, traveling, or vacationing.

Computer 12, as it designs a diet, will be capable of recognizing that each user 15 is different and has a unique body make-up. Every user 15 loses weight differently depending on a variety of factors, including activity, metabolism, age, gender, amount of weight to lose, and the number of diets they have been on in their lives. Through the central processing system 11, diet guidance portion of the software would be designed by a group of medical people, nutritionists, dieticians, diet counselors and experienced dieters.

Participant 15 will have an individualized weight control plan designed for him or her based on everyday foods. Variety is important to maintenance 26 of a dieter's interest in the system. Although it is important as part of the key to the success of a participant 15, it is also necessary to make sure a dieter 15 maintains their interest in the diet plan that they are following. Knowing that a wide variety of foods is available helps to provide an incentive for dieters 15.

For the participant that so chooses, there is a generic diet plan in processing system 11 available for any participant to follow. A dieter 15 may choose to follow their own diet plan or a plan specifically designed for them by their own doctor or their diet expert. Composite system 10 will allow dieter 15 to follow their own medically approved diet and still be able to participate in composite system 10 for regular weigh-in, the group therapy sessions and one-on-one counseling with all the benefits and privacy of the composite system and the program thereof

Other advantages and novel aspects of the invention will become apparent upon the following description in conjunction with the accompanying drawing wherein:

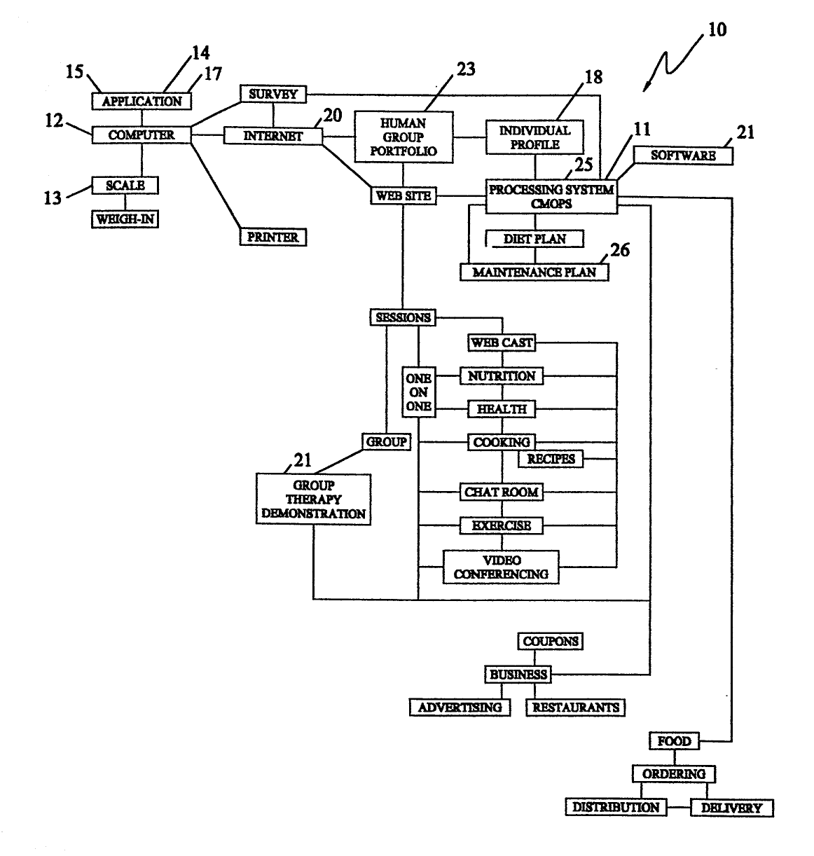

FIG. 1 is a schematic block diagram showing the interaction between elements of the invention and the subject user.

It is to be understood that the invention is not to be limited to the specific construction and arrangements shown and described, as it will be understood to those skilled in the art that certain changes may be made without departing from the principles of the invention.

What is claimed is:

1. A composite system for individual and group remote monitoring and directing of weight control procedures of certain human participant subject users comprising a computerized central processing system, said central processing system having a database system responsive to remote subject computer input for assembling input data, and a management processing system responsive to direct data management input, a remote subject computer unit linked to said central system, an electronic weight scale interfaced with said remote computer, said electronic weight scale interfaced with said subject computer and having a visual display for the benefit of a subject and being linked with said central system to visually indicate and forward, electronic weight readings to said central processing system to assemble an ongoing profile of the subject in said central processing system, and an application including identification and physiological profile of a given individual subject to be submitted to the composite system with said profile entered into said central processing system from said remote computer for data processing and management by said central processing system.

2. A composite system for individual and group remote monitoring and directing of weight control procedures of certain human subjects as defined in claim 1 wherein said central processing system has a software program to import and maintain said profile from said subject computer for individual feedback reading by the subject on a confidential one-on-one basis and on an anonymous comparative basis in a central chat portfolio group for reading by other subjects interfaced with said central system, and said central processing system having a software management program to directly import advice and counsel for individual feedback reading by the subject on a confidential one-on-one basis and on an anonymous comparative basis in a central portfolio chat group for reading by other subjects in said central processing system.

3. A composite system for individual and group remote monitoring and directing of weight control procedures of certain human subjects as defined in claim 2 wherein said central processing system software contains diet management data and advice specific to the subject system participant in regard to nutrition and health for the benefit of participant as well as other participants generally.

4. A composite system for individual and group remote monitoring and directing of weight control procedures of certain human subjects as defined in claim 2 wherein said central processing system software contains diet management data and advice specific to the subject system participant in regard to recipes and cooking procedures for the benefit of participant as well as other participants generally.

5. A composite system for individual and group remote monitoring and directing of weight control procedures of certain human subjects as defined in claim 2 wherein said central processing system software contains diet management data and advice specific to the subject system participant in regard to exercise for the benefit of participant as well as other participants generally.

6. A composite system for individual and group remote monitoring and directing of weight control procedures of certain human subjects as defined in claim 2 wherein said central processing system software contains diet management data and advice specific to the subject system participant in regard to video conferences for the benefit of participant as well as other participants generally.

7. A composite system for individual and group remote monitoring and directing of weight control procedures of certain human subjects as defined in claim 2 wherein said central processing system software contains diet management data and advice specific to the subject system participant in regard to library and chat room for the benefit of participant as well as other participants generally.

8. A composite system for individual and group remote monitoring and directing of weight control procedures of certain human subject as defined in claim 2 wherein said central processing system software contains diet management data and advice specific to the subject system participant in regard to group therapies for the benefit of participant as well as other participants generally.

9. A composite system for individual and group remote monitoring and directing of weight control procedures of certain human subjects as defined in claim 2 wherein said central processing system software contains diet management data and advice specific to the subject system participant in regard to available foods for the benefit of participant as well as other participants generally.

10. A composite system for individual and group remote monitoring and directing of weight control procedures of certain human subjects as defined in claim 3 wherein said central processing system software contains diet management data and advice specific to the subject system participant in regard to recipes and cooking procedures for the benefit of participant as well as other participants generally; and said central processing system software contains diet management data and advice specific to the subject system participant in regard to exercise for the benefit of participant as well as other participants generally.

11. A composite system for individual and group remote monitoring and directing of weight control procedures of certain human subjects as defined in claim 5 wherein said central processing system software contains diet management data and advice specific to the subject system participant in regard to video conferences for the benefit of participant as well as other participants generally; and said central processing system software contains diet management data and advice specific to the subject system participant in regard to library and chat room for the benefit of participant as well as other participants generally.

12. A composite system for individual and group remote monitoring and directing of weight control procedures of certain human subjects as defined in claim 7 wherein said central processing system software contains diet management data and advice specific to the subject system participant in regard to group therapies for the benefit of participant as well as other participants generally; and said central processing system software contains diet management data and advice specific to the subject system participant in regard to available foods for the benefit of participant as well as other participants generally.
